(19) United States
(12) Patent Application Publication
(10) Pub. No.: US 2024/0158838 A1
(43) Pub. Date: May 16, 2024

(54) METHODS OF MAKING GENE EXPRESSION LIBRARIES

(71) Applicant: 10x Genomics, Inc., Pleasanton, CA (US)

(72) Inventors: Luigi Jhon Alvarado Martinez, Gaithersburg, MD (US); Eswar Prasad Ramachandran Iyer, Oakland, CA (US)

(73) Assignee: 10x Genomics, Inc., Pleasanton, CA (US)

(21) Appl. No.: 18/418,967

(22) Filed: Jan. 22, 2024

Related U.S. Application Data
(63) Continuation of application No. 17/184,117, filed on Feb. 24, 2021, now Pat. No. 11,891,654.
(60) Provisional application No. 62/980,867, filed on Feb. 24, 2020.

(51) Int. Cl. C12Q 1/6837; C12Q 1/6816; C12Q 1/6827

(52) U.S. Cl. CPC C12Q 1/6837; C12Q 1/6816; C12Q 1/6827; C12Q 2543/101; C12Q 2565/514

(57) ABSTRACT

Provided herein are methods of determining a location of a target nucleic acid in a biological sample.

### CROSS-REFERENCE TO RELATED APPLICATIONS

This application is a continuation to U.S. patent application Ser. No. 17/184,117, filed Feb. 24, 2021, which claims priority to U.S. Provisional Patent Application Ser. No. 62/980,867, filed Feb. 24, 2020; the entire contents of which are herein incorporated by reference.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

### SUMMARY

This application is based on the discovery of a method of making a spatial 5′ gene expression library for spatial analysis of target analytes, including long target analytes e.g., VDJ rearranged T-cell receptors or immunoglobulins.

Provided herein are methods of identifying a location of a target nucleic acid in a permeabilized biological sample, the method comprising: (a) generating a cDNA molecule comprising a sequence that is substantially complementary to the target nucleic acid using a reverse transcription primer comprising (i) a sequence that is substantially complementary to a portion of the target nucleic acid and (ii) a first adaptor sequence, wherein the step of generating the cDNA molecule occurs within the permeabilized biological sample; (b) ligating a second adaptor sequence to a 3′ end of the cDNA molecule, wherein the step of ligating is performed within the biological sample; (c) after step (b), releasing the cDNA molecule from the target nucleic acid, such that the cDNA contacts an array, wherein the array comprises an attached capture probe comprising in a 5′ to a 3′ direction: (i) a spatial barcode and (ii) a capture domain that binds specifically to the second adaptor sequence ligated to the cDNA; (d) after step (c), extending a 3′ end of the capture probe using the cDNA as a template; and (e) determining (i) all or a part of the sequence of the target nucleic acid, or a complement thereof, and (ii) all or a part of the sequence of the spatial barcode, or a complement thereof, and using the determined sequences of (i) and (ii) to identify the location of the target nucleic acid in the permeabilized biological sample.

Also provided herein are methods of identifying a location of a target nucleic acid in a permeabilized biological sample, the method comprising: (a) generating a cDNA molecule comprising a sequence that is substantially complementary to the target nucleic acid using a reverse transcription primer comprising (i) a sequence that is substantially complementary to a portion of the target nucleic acid and (ii) a first adaptor sequence, wherein the step of generating the cDNA molecule occurs within the permeabilized biological sample; (b) extending a 3′ end of the cDNA molecule to include a second adaptor sequence, wherein the step of extending is performed within the biological sample; (c) releasing the cDNA molecule from the target nucleic acid, such that the cDNA contacts an array, wherein the array comprises an attached capture probe comprising in a 5′ to a 3′ direction: (i) a spatial barcode and (ii) a capture domain that binds specifically to the second adaptor sequence; (d) extending a 3′ end of the capture probe using the cDNA as a template; and (c) determining (i) all or a part of the sequence of the target nucleic acid, or a complement thereof, and (ii) the sequence of the spatial barcode, or a complement thereof, and using the determined sequences of (i) and (ii) to identify the location of the target nucleic acid in the permeabilized biological sample. In some embodiments, step (b) can occur simultaneously with step (a).

In some embodiments of any of the methods described herein, steps (a) through (c) are performed when the biological sample is disposed on the array.

In some embodiments of any of the methods described herein, step (a) is performed when the biological sample is not disposed on the array and step (b) is performed when the biological sample is disposed on the array, and wherein the method further comprises between steps (a) and (b), a step of disposing the biological sample on the array.

In some embodiments of any of the methods described herein, steps (a) and (b) are performed when the biological sample is not disposed on the array, and wherein the method further comprises between steps (b) and (c), a step of disposing the biological sample on the array.

In some embodiments of any of the methods described herein, the sequence that is substantially complementary to a portion of the target nucleic acid present in the reverse transcription primer comprises a poly(T) sequence.

In some embodiments of any of the methods described herein, the sequence that is substantially complementary to a portion of the target nucleic acid present in the reverse transcription primer comprises a random sequence.

In some embodiments of any of the methods described herein, the second adaptor sequence is a template switching oligonucleotide (TSO).

In some embodiments of any of the methods described herein, the array comprises a slide.

In some embodiments of any of the methods described herein, a 5′ end of the capture probe is attached to the slide.

In some embodiments of any of the methods described herein, the array is a bead array.

In some embodiments of any of the methods described herein, a 5′ end of the capture probe is attached to a bead of the bead array.

In some embodiments of any of the methods described herein, the capture probe further comprises a unique molecular identifier (UMI).

In some embodiments of any of the methods described herein, the UMI is positioned 5′ relative to the capture domain in the capture probe.

In some embodiments of any of the methods described herein, the determining in step (e) comprises sequencing (i) all or a part of the sequence of the target nucleic acid, or a complement thereof, and (ii) all or a part of the sequence of the spatial barcode, or a complement thereof.

In some embodiments of any of the methods described herein, the sequencing is high throughput sequencing.

In some embodiments of any of the methods described herein, the sequencing is sequencing by hybridization.

In some embodiments of any of the methods described herein, the target nucleic acid is RNA.

In some embodiments of any of the methods described herein, the RNA is an mRNA.

In some embodiments of any of the methods described herein, the permeabilized biological sample is a permeabilized tissue section.

In some embodiments of any of the methods described herein, the permeabilized tissue section is a permeabilized formalin-fixed and paraffin-embedded (FFPE) tissue section.

Some embodiments of any of the methods described herein further comprises a step of imaging the biological sample.

In some embodiments of any of the methods described herein, the step of imaging is performed prior to step (a).

In some embodiments of any of the methods described herein, the step of imaging is performed between steps (b) and (c).

Some embodiments of any of the methods described herein further comprises, between steps (b) and (c), a step of freezing and thawing the permeabilized biological sample.

Some embodiments of any of the methods described herein further comprises, between steps (b) and (c), a step of sectioning the permeabilized biological sample.

In some embodiments of any of the methods described herein, the step of sectioning the permeabilized biological sample is performed using cryosectioning.

Some embodiments of any of the methods described herein further comprises, prior to step (a), a step of permeabilizing the biological sample.

In some embodiments of any of the methods described herein, the performance of step (a) comprises introducing a reverse transcriptase, dNTPs, and the reverse transcription primer into the permeabilized biological sample.

In some aspects, provided herein are kits comprising: a reverse transcription primer comprising (i) a sequence that is substantially complementary to a portion of the target nucleic acid and (ii) a first adaptor sequence; a reverse transcriptase; and an oligonucleotide comprising a second adaptor sequence or a complement thereof.

In some embodiments of any of the kits provided herein, the kit further comprises a ligase.

In some embodiments of any of the kits provided herein, the reverse transcriptase is a reverse transcriptase with terminal transferase activity.

In some embodiments of any of the kits provided herein, the second adaptor sequence or the complement thereof is a TSO or a complement thereof.

In some embodiments of any of the kits provided herein, the kit further comprises an array, wherein the array comprises an attached capture probe comprising in a 5′ to a 3′ direction: (i) a spatial barcode and (ii) a capture domain that binds specifically to the second adaptor sequence.

In another aspect, provided herein are nucleic acids comprising, in the 5′ to 3′ direction: a spatial barcode; a sequence complementary to a second adaptor sequence; a sequence present in a target nucleic acid; and a sequence complementary to a first adaptor sequence.

In another aspect, provided herein are nucleic acids comprising, in the 3′ to 5′ direction: a complement of a spatial barcode; a second adaptor sequence; a sequence complementary to a sequence present in a target nucleic acid; and a first adaptor sequence.

In some embodiments of any of the nucleic acids provided herein, the second adaptor sequence comprises a TSO.

In some embodiments of any of the nucleic acids provided herein, the second adaptor sequence comprises a complement of a TSO.

In some embodiments of any of the nucleic acids provided herein, the first adaptor sequence is a reverse transcriptase primer.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise. Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows a workflow schematic illustrating exemplary, non-limiting steps for in-situ cDNA synthesis and capturing.

FIG. 1B shows a workflow schematic illustrating exemplary, non-limiting steps for building a 5′ spatial gene expression library.

FIG. 2A shows a workflow schematic illustrating exemplary, non-limiting steps for in-situ cDNA synthesis and sample handling.

FIG. 2B shows a workflow schematic illustrating exemplary, non-limiting steps for building a 5′ spatial gene expression library.

FIG. 3A shows a workflow schematic illustrating exemplary, non-limiting steps for synthesizing a cDNA molecule.

FIG. 3B shows a workflow schematic illustrating exemplary, non-limiting steps for cDNA binding to an attached capture probe and the extension of the capture probe using the cDNA molecule as a template, and the generation of a second strand complementary to the extended capture probe.

FIG. 3C shows a workflow schematic illustrating exemplary, non-limiting, non-exhaustive steps for building a 5′ spatial gene expression library.

### DETAILED DESCRIPTION

In some cases, spatial analysis methods can be carried out by permeabilizing a biological sample, capturing analytes (e.g., nucleic acids (e.g., mRNA)) or intermediate agents on an array, and performing reverse transcription and sequencing steps to identify the location of one or more analytes from the biological sample. Many capture protocols rely on the use of a poly(A) tail, either natural or introduced. Several challenges can arise from these protocols. For example, there may or may not be biased in the analytes or intermediate agents that are able to migrate from a biological sample to the array. As another example, capture by the poly(A) tail can lead to a 3′ bias in gene expression libraries generated from mRNAs due to limitations of some steps of the process. Provided herein are methods that can, in some cases, address one or both of these challenges.

Provided herein are methods of identifying a location of a target nucleic acid in a permeabilized biological sample, the method comprising: (a) generating a cDNA molecule comprising a sequence that is substantially complementary to the target nucleic acid using a reverse transcription primer comprising (i) a sequence that is substantially complementary to a portion of the target nucleic acid and (ii) a first adaptor sequence, wherein the step of generating the cDNA molecule occurs within the permeabilized biological sample; (b) ligating a second adaptor sequence to a 3′ end of the cDNA molecule, wherein the step of ligating is performed within the biological sample; (c) after step (b), releasing the cDNA molecule from the target nucleic acid, such that the cDNA contacts an array, wherein the array comprises an attached capture probe comprising in a 5′ to a 3′ direction: (i) a spatial barcode and (ii) a capture domain that binds specifically to the second adaptor sequence ligated to the cDNA; (d) after step (c), extending a 3′ end of the capture probe using the cDNA as a template; and (e) determining (i) all or a part of the sequence of the target nucleic acid, or a complement thereof, and (ii) all or a part of the sequence of the spatial barcode, or a complement thereof, and using the determined sequences of (i) and (ii) to identify the location of the target nucleic acid in the permeabilized biological sample. Non-limiting aspects of these methods are described herein.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434): 1463-1467, 2019; Lee et al., Nat. Protoc. 10(3): 442-458, 2015; Trejo et al., PLOS ONE 14(2): c0212031, 2019; Chen et al., Science 348(6233): aaa6090, 2015; Gao et al., BMC Biol. 15: 50, 2017; and Gupta et al., Nature Biotechnol. 36: 1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture domain can include a sequence that is significantly complementary to an analyte, a complement thereof, or a portion thereof (e.g., a capture domain can include a poly-T sequence). In some embodiments, a capture domain can include a sequence that is significantly complementary to a sequence introduced to the analyte before capture (e.g., a capture domain can include a sequence complementary to a functional domain and/or an adaptor sequence (e.g., a template switching oligonucleotide sequence)). In some embodiments, a capture probe can include a cleavage domain and/or a functional domain (e.g., a primer-binding site, such as for next-generation sequencing (NGS)). Sec, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, genetic material is amplified by reverse transcription polymerase chain reaction (RT-PCR). The desired reverse transcriptase activity can be provided by one or more distinct reverse transcriptase enzymes (i.e., RNA dependent DNA polymerases), suitable examples of which include, but are not limited to: M-MLV, MuLV, AMV, HIV, ArrayScript™, MultiScribe™, ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g., reverse transcriptase, activity of the wild-type sequence. The reverse transcriptase enzyme can be provided as part of a composition that includes other components, e.g., stabilizing components that enhance or improve the activity of the reverse transcriptase enzyme, such as RNase inhibitor(s), inhibitors of DNA-dependent DNA synthesis, e.g., actinomycin D. Many sequence-modified derivative or mutants of reverse transcriptase enzymes, e.g., M-MLV, and compositions including unmodified and modified enzymes are commercially available, e.g., ArrayScript™, MultiScribe™, ThermoScript™, and SuperScript® I, II, III, and IV enzymes.

Certain reverse transcriptase enzymes (e.g., Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g., an AMV or MMLV reverse transcriptase.

In some embodiments, the quantification of RNA and/or DNA is carried out by real-time PCR (also known as quantitative PCR or qPCR), using techniques well known in the art, such as but not limited to “TAQMAN™”, or dyes such as “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

A “template switching oligonucleotide” (TSO) is an oligonucleotide that hybridizes to untemplated nucleotides added by a reverse transcriptase (e.g., enzyme with terminal transferase activity) during reverse transcription. In some embodiments, a template switching oligonucleotide hybridizes to untemplated poly(C) nucleotides added by a reverse transcriptase. In some embodiments, the template switching oligonucleotide adds a common 5′ sequence to full-length cDNA that is used for cDNA amplification.

In some embodiments, the template switching oligonucleotide adds a common sequence onto the 5′ end of the RNA being reverse transcribed. For example, a template switching oligonucleotide can hybridize to untemplated poly(C) nucleotides added onto the end of a cDNA molecule and provide a template for the reverse transcriptase to continue replication to the 5′ end of the template switching oligonucleotide, thereby generating full-length cDNA ready for further amplification. In some embodiments, once a full-length cDNA molecule is generated, the template switching oligonucleotide can serve as a primer in a cDNA amplification reaction.

In some embodiments, a template switching oligonucleotide is added before, contemporaneously with, or after a reverse transcription, or other terminal transferase-based reaction. In some embodiments, a template switching oligonucleotide or complement thereof is included in the capture probe. In some embodiments, the TSO, or complement thereof, in the capture probe serves as a capture domain. In certain embodiments, methods of sample analysis using template switching oligonucleotides can involve the generation of nucleic acid products from analytes of the tissue sample, followed by further processing of the nucleic acid products with the template switching oligonucleotide.

Template switching oligonucleotides can include a hybridization region and a template region. The hybridization region can include any sequence capable of hybridizing to the target sequence. In some embodiments, the hybridization region can, e.g., include a series of G bases to complement the overhanging C bases at the 3′ end of a cDNA molecule. The series of G bases can include 1 G base, 2 G bases, 3 G bases, 4 G bases, 5 G bases, or more than 5 G bases. The template sequence can include any sequence to be incorporated into the cDNA. In other embodiments, the hybridization region can include at least one base in addition to at least one G base. In other embodiments, the hybridization can include bases that are not a G base. In some embodiments, the template region includes at least 1 (e.g., at least 2, 3, 4, 5 or more) tag sequences and/or functional sequences. In some embodiments, the template region and hybridization region are separated by a spacer.

In some embodiments, the template regions include a barcode sequence. The barcode sequence can act as a spatial barcode and/or as a unique molecular identifier. In some embodiments, the template region can include a functional region, for example a region that can be used for amplification, a region that is complementary to a capture domain on a capture probe, etc. In some embodiments, the template region can include a barcode and/or a unique molecular identifier and/or a functional sequence and/or a capture domain sequence. Template switching oligonucleotides can include deoxyribonucleic acids; ribonucleic acids; modified nucleic acids including 2-aminopurine, 2,6-diaminopurine (2-amino-dA), inverted dT, 5-methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ fluoro bases (e.g., Fluoro C, Fluoro U, Fluoro A, and Fluoro G), or any combination of the foregoing.

In some embodiments, the length of a template switching oligonucleotide can be at least about 1, 2, 10, 20, 50, 75, 100, 150, 200, or 250 nucleotides or longer. In some embodiments, the length of a template switching oligonucleotide can be at most about 2, 10, 20, 50, 100, 150, 200, or 250 nucleotides or longer.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligation products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. Sec, e.g., Credle et al., Nucleic Acids Res. 2017 Aug 21; 45(14): e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(c)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or scalable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### Spatial 5′ Gene Expression Libraries

Provided herein are methods of identifying a location of a target nucleic acid in a permeabilized biological sample, the method comprising: (a) generating a cDNA molecule comprising a sequence that is substantially complementary to the target nucleic acid using a reverse transcription primer comprising (i) a sequence that is substantially complementary to a portion of the target nucleic acid and (ii) a first adaptor sequence, wherein the step of generating the cDNA molecule occurs within the permeabilized biological sample; (b) ligating a second adaptor sequence to a 3′ end of the cDNA molecule, wherein the step of ligating is performed within the biological sample; (c) releasing the cDNA molecule from the target nucleic acid, such that the cDNA contacts an array, wherein the array comprises an attached capture probe comprising in a 5′ to a 3′ direction: (i) a spatial barcode and (ii) a capture domain that binds specifically to the second adaptor sequence ligated to the cDNA; (d) extending a 3′ end of the capture probe using the cDNA as a template; and (e) determining (i) all or a part of the sequence of the target nucleic acid, or a complement thereof, and (ii) all or a part of the sequence of the spatial barcode, or a complement thereof, and using the determined sequences of (i) and (ii) to identify the location of the target nucleic acid in the permeabilized biological sample.

Also provided herein are methods of identifying a location of a target nucleic acid in a permeabilized biological sample, the method comprising: (a) generating a cDNA molecule comprising a sequence that is substantially complementary to the target nucleic acid using a reverse transcription primer comprising (i) a sequence that is substantially complementary to a portion of the target nucleic acid and (ii) a first adaptor sequence, wherein the step of generating the cDNA molecule occurs within the permeabilized biological sample; (b) extending a 3′ end of the cDNA molecule to include a second adaptor sequence, wherein the step of extending is performed within the biological sample; (c) releasing the cDNA molecule from the target nucleic acid, such that the cDNA contacts an array, wherein the array comprises an attached capture probe comprising in a 5′ to a 3′ direction: (i) a spatial barcode and (ii) a capture domain that binds specifically to the second adaptor sequence ligated to the cDNA; (d) extending a 3′ end of the capture probe using the cDNA as a template; and (e) determining (i) all or a part of the sequence of the target nucleic acid, or a complement thereof, and (ii) all or a part of the sequence of the spatial barcode, or a complement thereof, and using the determined sequences of (i) and (ii) to identify the location of the target nucleic acid in the permeabilized biological sample. In some such embodiments, the method can further comprise hybridizing a template switching oligonucleotide (TSO) to the cDNA molecule. Therefore, in some embodiments, (b) comprises extending a 3′ end of the cDNA molecule to include a complement of a TSO. In some cases, the TSO can be added to the sample at the same time as the reverse transcriptase primer.

In some embodiments, steps (a) through (c) are performed when the biological sample is disposed on the array. In some embodiments, step (a) is performed when the biological sample is not disposed on the array and step (b) is performed when the biological sample is disposed on the array, and wherein the method further comprises between steps (a) and (b), a step of disposing the biological sample on the array. In some embodiments, steps (a) and (b) are performed when the biological sample is not disposed on the array, and wherein the method further comprises between steps (b) and (c), a step of disposing the biological sample on the array.

In some embodiments of any of the methods described herein, the biological sample can be any of the exemplary permeabilized biological samples described herein (e.g., a permeabilized tissue sample, e.g., a permeabilized permeabilized tissue section), or any of the same described in, e.g., Section (I)(d) (e.g., (I)(d)(i) and/or (I)(d)(ii)(13)) of WO 2020/176788 and/or U.S. Patent Application Publication No. 2020/0277663. Some embodiments described herein can optionally further include a step of permeabilizing the biological sample (e.g., using any of the exemplary methods and agents for permeabilizing a biological sample described herein). In some embodiments, the target nucleic acid is RNA (e.g., mRNA).

In some embodiments, the reverse transcription primer can have a total of about 10 nucleotides to about 250 nucleotides (e.g., about 10 nucleotides to about 225 nucleotides, about 10 nucleotides to about 200 nucleotides, about 10 nucleotides to about 175 nucleotides, about 10 nucleotides to about 150 nucleotides, about 10 nucleotides to about 125 nucleotides, about 10 nucleotides to about 100 nucleotides, about 10 nucleotides to about 80 nucleotides, about 10 nucleotides to about 60 nucleotides, about 10 nucleotides to about 40 nucleotides, about 10 nucleotides to about 20 nucleotides, about 20 nucleotides to about 250 nucleotides, about 20 nucleotides to about 225 nucleotides, about 20 nucleotides to about 200 nucleotides, about 20 nucleotides to about 175 nucleotides, about 20 nucleotides to about 150 nucleotides, about 20 nucleotides to about 125 nucleotides, about 20 nucleotides to about 100 nucleotides, about 20 nucleotides to about 80 nucleotides, about 20 nucleotides to about 60 nucleotides, about 20 nucleotides to about 40 nucleotides, about 40 nucleotides to about 250 nucleotides, about 40 nucleotides to about 225 nucleotides, about 40 nucleotides to about 200 nucleotides, about 40 nucleotides to about 175 nucleotides, about 40 nucleotides to about 150 nucleotides, about 40 nucleotides to about 125 nucleotides, about 40 nucleotides to about 100 nucleotides, about 40 nucleotides to about 80 nucleotides, about 40 nucleotides to about 60 nucleotides, about 60 nucleotides to about 250 nucleotides, about 60 nucleotides to about 225 nucleotides, about 60 nucleotides to about 200 nucleotides, about 60 nucleotides to about 175 nucleotides, about 60 nucleotides to about 150 nucleotides, about 60 nucleotides to about 125 nucleotides, about 60 nucleotides to about 100 nucleotides, about 60 nucleotides to about 80 nucleotides, about 80 nucleotides to about 250 nucleotides, about 80 nucleotides to about 225 nucleotides, about 80 nucleotides to about 200 nucleotides, about 80 nucleotides to about 175 nucleotides, about 80 nucleotides to about 150 nucleotides, about 80 nucleotides to about 125 nucleotides, about 80 nucleotides to about 100 nucleotides, about 100 nucleotides to about 250 nucleotides, about 100 nucleotides to about 225 nucleotides, about 100 nucleotides to about 200 nucleotides, about 100 nucleotides to about 175 nucleotides, about 100 nucleotides to about 150 nucleotides, about 100 nucleotides to about 125 nucleotides, about 125 nucleotides to about 250 nucleotides, about 125 nucleotides to about 225 nucleotides, about 125 nucleotides to about 200 nucleotides, about 125 nucleotides to about 175 nucleotides, about 125 nucleotides to about 150 nucleotides, about 150 nucleotides to about 250 nucleotides, about 150 nucleotides to about 225 nucleotides, about 150 nucleotides to about 200 nucleotides, about 150 nucleotides to about 175 nucleotides, about 175 nucleotides to about 250 nucleotides, about 175 nucleotides to about 225 nucleotides, about 175 nucleotides to about 200 nucleotides, about 200 nucleotides to about 250 nucleotides, about 200 nucleotides to about 225 nucleotides, or about 225 nucleotides to about 250 nucleotides).

In some embodiments of any of the methods described herein, the first adaptor sequence has a total of about 5 nucleotides to about 125 nucleotides (e.g., about 5 nucleotides to about 100 nucleotides, about 5 nucleotides to about 90 nucleotides, about 5 nucleotides to about 80 nucleotides, about 5 nucleotides to about 70 nucleotides, about 5 nucleotides to about 60 nucleotides, about 5 nucleotides to about 50 nucleotides, about 5 nucleotides to about 45 nucleotides, about 5 nucleotides to about 40 nucleotides, about 5 nucleotides to about 35 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides, about 10 nucleotides to about 125 nucleotides, about 10 nucleotides to about 100 nucleotides, about 10 nucleotides to about 90 nucleotides, about 10 nucleotides to about 80 nucleotides, about 10 nucleotides to about 70 nucleotides, about 10 nucleotides to about 60 nucleotides, about 10 nucleotides to about 50 nucleotides, about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 25 nucleotides, about 10 nucleotides to about 20 nucleotides, about 10 nucleotides to about 15 nucleotides, about 20 nucleotides to about 125 nucleotides, about 20 nucleotides to about 100 nucleotides, about 20 nucleotides to about 90 nucleotides, about 20 nucleotides to about 80 nucleotides, about 20 nucleotides to about 70 nucleotides, about 20 nucleotides to about 60 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 25 nucleotides, about 30 nucleotides to about 125 nucleotides, about 30 nucleotides to about 100 nucleotides, about 30 nucleotides to about 90 nucleotides, about 30 nucleotides to about 80 nucleotides, about 30 nucleotides to about 70 nucleotides, about 30 nucleotides to about 60 nucleotides, about 30 nucleotides to about 50 nucleotides, about 30 nucleotides to about 45 nucleotides, about 30 nucleotides to about 40 nucleotides, about 30 nucleotides to about 35 nucleotides, about 40 nucleotides to about 125 nucleotides, about 40 nucleotides to about 100 nucleotides, about 40 nucleotides to about 90 nucleotides, about 40 nucleotides to about 80 nucleotides, about 40 nucleotides to about 70 nucleotides, about 40 nucleotides to about 60 nucleotides, about 40 nucleotides to about 50 nucleotides, about 40 nucleotides to about 45 nucleotides, about 50 nucleotides to about 125 nucleotides, about 50 nucleotides to about 100 nucleotides, about 50 nucleotides to about 90 nucleotides, about 50 nucleotides to about 80 nucleotides, about 50 nucleotides to about 70 nucleotides, about 50 nucleotides to about 60 nucleotides, about 60 nucleotides to about 125 nucleotides, about 60 nucleotides to about 100 nucleotides, about 60 nucleotides to about 90 nucleotides, about 60 nucleotides to about 80 nucleotides, about 60 nucleotides to about 70 nucleotides, about 70 nucleotides to about 125 nucleotides, about 70 nucleotides to about 100 nucleotides, about 70 nucleotides to about 90 nucleotides, about 70 nucleotides to about 80 nucleotides, about 80 nucleotides to about 125 nucleotides, about 80 nucleotides to about 100 nucleotides, about 80 nucleotides to about 90 nucleotides, about 90 nucleotides to about 125 nucleotides, about 90 nucleotides to about 100 nucleotides, or about 100 nucleotides to about 125 nucleotides). In some embodiments, the first adaptor sequence can be any predetermined sequence. In some embodiments, the first adaptor sequence does not encode a polypeptide and/or can be non-naturally occurring sequence.

In some embodiments the sequence that is substantially complementary (e.g., at least 80%, at least 85%, at least 90%, at least 95%, at least 99%, or 100% complementary) to a portion of the sequence of the target nucleic acid (that is present in the reverse transcription primer) can have a total of about 10 nucleotides to about 125 nucleotides (or any of the subranges of this range described herein). In some embodiments, the sequence that is substantially complementary to a portion of the sequence of the target nucleic acid can be a random sequence. In some embodiments, the sequence that is substantially complementary to a portion of the sequence of the target nucleic acid can include a poly(T) oligonucleotide sequence (e.g., at least 5 contiguous Ts, at least 10 continguous Ts, or at least 15 contiguous Ts).

In some embodiments, the step of generating a cDNA molecule including a sequence that is substantially complementary to the target nucleic acid using a reverse transcription primer can include contacting a permeabilized biological sample with a reverse transcriptase (e.g., any of the exemplary reverse transcriptases described herein or known in the art), dNTPs, and the reverse transcription primer (e.g., any of the exemplary reverse transcription primers described herein). A variety of kits including a reverse transcriptase and dNTPs are commercially available. Non-limiting examples of conditions for generating a cDNA molecule are described herein, and additional examples of conditions for generating a cDNA molecule are known in the art.

In some embodiments of any of the methods described herein, the second adaptor sequence (e.g., ligated to a 3′ end of the generated cDNA molecule (performed within the biological sample), or included in the generated cDNA molecule via extension of a 3′ end of the cDNA molecule) can have a total of about 5 nucleotides to about 125 nucleotides (or any of the subranges of this range described herein). In some embodiments, the second adaptor sequence can be any predetermined sequence. In some embodiments, the second adaptor sequence does not encode a polypeptide and/or can be non-naturally occurring sequence. In some embodiments, the first and second adaptor sequences include different sequences. In some embodiments, the second adaptor sequence can be a template switching oligonucleotide (TSO) (e.g., any of the exemplary TSOs described herein), or a complement thereof. In some embodiments, the second adaptor sequence includes a sequence that is substantially complementary (e.g., at least 80%, at least 85%, at least 90%, at least 92%, at least 94%, at least 96%, at least 98%, at least 99%, or 100% complementary) to a sequence in the capture domain of the capture probe.

In some embodiments, the step of ligating the second adaptor sequence to a 3′ end of the generated cDNA molecule (performed within the biological sample) can be performed using any of the ligation methods described herein or known in the art. A wide variety of different methods can be used for ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end ligations generally do not include hybridization of complementary regions from each nucleic acid molecule because both nucleic acid molecules lack a single-stranded overhang at the site of ligation. In some embodiments, DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oNTM DNA ligase, available from New England Biolabs, Ipswich, MA), and Ampligase® (available from Lucigen, Middleton, WI). Derivatives, e.g., sequence-modified derivatives, and/or mutants thereof, can also be used.

For example, the step of ligating can be performed by contacting the permeabilized biological sample with a ligase and the second adaptor sequence (e.g., a TSO), and optionally, any additional components required to accelerate the ligation reaction. In some embodiments, the methods can further include blocking the 5′ end of the generated cDNA molecule prior to the ligating step. Non-limiting examples of conditions for performing ligation are described herein, and additional examples of conditions for performing ligation are known in the art.

In some embodiments, extension of a 3′ end of a generated cDNA to include a second adaptor sequence can be performed using any appropriate methods, such as those described herein for a TSO. For example, the step of extension of a 3′ end of a generated cDNA molecule can include hybridizing an oligo comprising a complement of the second adaptor sequence to a portion of a 3′ end of the generated cDNA molecule, and extending the cDNA molecule to include the second adaptor sequence. In some cases, the terminal transferase activity of a reverse transcriptase enzyme will result in a polymononucleotide sequence (e.g., a poly(C) sequence) at the 3′ end of a generated cDNA molecule, and the oligo comprising a complement of the second adaptor sequence can further include a complementary polymononucleotide sequence (e.g., a poly(G) sequence) that allows for hybridization to the polymononucleotide sequence of the cDNA molecule. The hybridized oligo comprising the complement of the second adaptor sequence can then be used as a template for extending the 3′ end of the generated cDNA molecule to include the second adaptor sequence. In some cases, the oligo comprising a complement of the second adaptor sequence is added to the sample at the same time as the reverse transcription primer.

In some embodiments, the releasing of the cDNA molecule from the target nucleic acid can be performed by using heat or a chemical denaturant (e.g., KOH).

In some embodiments of any of the methods described herein, the array can be any of the types of arrays described herein. For example, the array includes a slide. In some embodiments, the capture probe is attached to the slide (e.g., by its 5′ end).

In some embodiments, the array is a bead array. In some embodiments, a 5′ end of the capture probe is attached to a bead of the bead array.

In some embodiments, the capture probe further comprises a unique molecular identifier (UMI) (e.g., a UMI positioned 5′ relative to the capture domain the capture probe).

In some embodiments, the determining in step (e) comprises sequencing (i) all or a part of the sequence of the target nucleic acid, or a complement thereof, and (ii) all or a part of the sequence of the spatial barcode, or a complement thereof. In some embodiments, the sequencing is high throughput sequencing, sequencing by hybridization, or any of the other methods for sequencing described herein or known in the art. For example, sequencing can involve one or more of nucleic acid amplification, the ligation or addition of one or more sequencing adaptors, cleavage of the capture probe from the array, extension of the capture probe using the bound cDNA as a template, and generating a single-stranded nucleic acid comprising a sequence that is complementary to the extended capture probe. Non-limiting methods for determining the sequence of (i) all or a part of the sequence of the target nucleic acid, or a complement thereof, or (ii) all or a part of the sequence of the spatial barcode, or a complement thereof, are described herein or are known in the art.

In some embodiments, the methods can optionally further include a step of imaging the biological sample (e.g., using any of the exemplary imaging methods described herein or known in the art). In some embodiments, the imaging is performed prior to step (a). In some embodiments, the imaging is performed between steps (b) and (c).

In some embodiments, the method further includes, between steps (b) and (c), a step of freezing and thawing the permeabilized biological sample. In some embodiments, the method can further include, between steps (b) and (c), a step of sectioning (e.g., cryosectioning) the permeabilized biological sample.

### Exemplary Embodiments

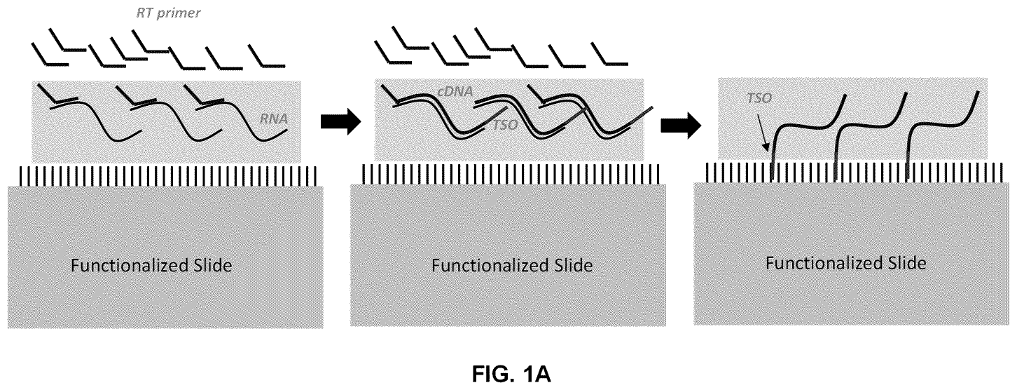

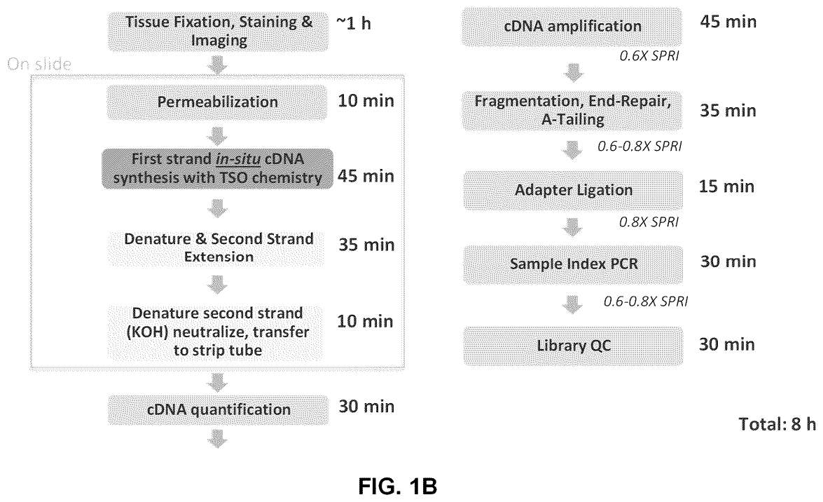

FIG. 1A is an exemplary diagram showing, from left to right, the hybridization of a reverse transcription primer to a target nucleic acid, e.g., an mRNA, within a permeabilized biological sample; the generation of a cDNA molecule comprising a sequence that is substantially complementary to the target nucleic acid, and the addition/ligation of a second adaptor sequence (e.g., a template switching oligonucleotide, or a complement thereof) to the 3′ end of the cDNA molecule within the permeabilized biological sample; and releasing of the cDNA molecule from the target nucleic acid and contacting the released cDNA molecule to an array (e.g., any of the exemplary arrays described herein) comprising a capture probe for performance of additional steps (e.g., any of the exemplary additional steps described herein). The second adaptor sequence added (e.g., via extension)/ligated to the cDNA specifically binds to the capture probe.

FIG. 1B is an exemplary workflow showing the generation of a gene expression library and the identification of the location of a target nucleic acid in a biological sample, for example following a target analyte capture workflow as shown pictorially in FIG. 1A. Specifically, a biological sample, e.g., a tissue sample, is fixed, stained, and imaged. Any of the exemplary methods described herein or known in the art can be used to fix, stain, and/or image the biological sample. In some embodiments, the biological sample can be a formalin-fixed and paraffin-embedded (FFPE) tissue sample. In some embodiments, the biological sample is stained, e.g., using an H&E staining method. In some embodiments, the tissue sample is fixed, stained, and/or imaged for 5 minutes to about 5 hours, e.g., about 5 minutes to about 4.5 hours, about 5 minutes to about 4.0 hours, about 5 minutes to about 3.5 hours, about 5 minutes to about 3.0 hours, about 5 minutes to about 2.5 hours, about 5 minutes to about 2.0 hours, about 5 minutes to about 1.5 hours, about 5 minutes to about 1.0 hour, about 5 minutes to about 50 minutes, about 5 minutes to about 40 minutes, about 5 minutes to about 30 minutes, about 5 minutes to about 20 minutes, about 5 minutes to about 10 minutes, about 10 minutes to about 5 hours, about 10 minutes to about 4.5 hours, about 10 minutes to about 4.0 hours, about 10 minutes to about 3.5 hours, about 10 minutes to about 3.0 hours, about 10 minutes to about 2.5 hours, about 10 minutes to about 2.0 hours, about 10 minutes to about 1.5 hours, about 10 minutes to about 1.0 hour, about 10 minutes to about 50 minutes, about 10 minutes to about 40 minutes, about 10 minutes to about 30 minutes, about 10 minutes to about 20 minutes, about 20 minutes to about 5 hours, about 20 minutes to about 4.5 hours, about 20 minutes to about 4.0 hours, about 20 minutes to about 3.5 hours, about 20 minutes to about 3.0 hours, about 20 minutes to about 2.5 hours, about 20 minutes to about 2.0 hours, about 20 minutes to about 1.5 hours, about 20 minutes to about 1.0 hour, about 20 minutes to about 50 minutes, about 20 minutes to about 40 minutes, about 20 minutes to about 30 minutes, about 30 minutes to about 5 hours, about 30 minutes to about 4.5 hours, about 30 minutes to about 4.0 hours, about 30 minutes to about 3.5 hours, about 30 minutes to about 3.0 hours, about 30 minutes to about 2.5 hours, about 30 minutes to about 2.0 hours, about 30 minutes to about 1.5 hours, about 30 minutes to about 1.0 hour, about 30 minutes to about 50 minutes, about 30 minutes to about 40 minutes, about 1.0 hour to about 5 hours, about 1.0 hour to about 4.5 hours, about 1.0 hour to about 4.0 hours, about 1.0 hour to about 3.5 hours, about 1.0 hour to about 3.0 hours, about 1.0 hour to about 2.5 hours, about 1.0 hour to about 2.0 hours, about 1.0 hour to about 1.5 hours, about 1.5 hour to about 5 hours, about 1.5 hour to about 4.5 hours, about 1.5 hour to about 4.0 hours, about 1.5 hour to about 3.5 hours, about 1.5 hour to about 3.0 hours, about 1.5 hour to about 2.5 hours, about 1.5 hour to about 2.0 hours, about 2.0 hour to about 5 hours, about 2.0 hour to about 4.5 hours, about 2.0 hour to about 4.0 hours, about 2.0 hour to about 3.5 hours, about 2.0 hour to about 3.0 hours, about 2.0 hour to about 2.5 hours, about 2.5 hour to about 5 hours, about 2.5 hour to about 4.5 hours, about 2.5 hour to about 4.0 hours, about 2.5 hour to about 3.5 hours, about 2.5 hour to about 3.0 hours, about 3.0 hour to about 5 hours, about 3.0 hour to about 4.5 hours, about 3.0 hour to about 4.0 hours, about 3.0 hour to about 3.5 hours, about 3.5 hour to about 5 hours, about 3.5 hour to about 4.5 hours, about 3.5 hour to about 4.0 hours, about 4.0 hour to about 5 hours, about 4.0 hour to about 4.5 hours, or about 4.5 hour to about 5 hours.

After the fixation, staining and imaging of the biological sample, the biological sample is permeabilized. Permeabilization of the biological sample (e.g., a tissue sample) can be performed using any of the exemplary methods or exemplary reagents described herein, or in, e.g., Section (I)(d)(ii)(13) of WO 2020/176788 and/or U.S. Patent Application Publication No. 2020/0277663. In some embodiments, the permeabilization of the biological sample (e.g., tissue sample) can be performed for about 1 minute to about 5 hours (e.g., about 1 minute to about 4.5 hours, about 1 minute to about 4.0 hours, about 1 minute to about 3.5 hours, about 1 minute to about 3.0 hours, about 1 minute to about 2.5 hours, about 1 minute to about 2.0 hours, about 1 minute to about 1.5 hours, about 1 minute to about 1.0 hour, about 1 minute to about 50 minutes, about 1 minute to about 40 minutes, about 1 minute to about 30 minutes, about 1 minute to about 20 minutes, about 1 minute to about 10 minutes, about 1 minute to about 5 minutes, about 10 minutes to about 5.0 hours, about 10 minutes to about 4.5 hours, about 10 minutes to about 4.0 hours, about 10 minutes to about 3.5 hours, about 10 minutes to about 3.0 hours, about 10 minutes to about 2.5 hours, about 10 minutes to about 2.0 hours, about 10 minutes to about 1.5 hours, about 10 minutes to about 1.0 hour, about 10 minutes to about 50 minutes, about 10 minutes to about 40 minutes, about 10 minutes to about 30 minutes, about 10 minutes to about 20 minutes, about 20 minutes to about 5.0 hours, about 20 minutes to about 4.5 hours, about 20 minutes to about 4.0 hours, about 20 minutes to about 3.5 hours, about 20 minutes to about 3.0 hours, about 20 minutes to about 2.5 hours, about 20 minutes to about 2.0 hours, about 20 minutes to about 1.5 hours, about 20 minutes to about 1.0 hour, about 20 minutes to about 50 minutes, about 20 minutes to about 40 minutes, about 20 minutes to about 30 minutes, about 30 minutes to about 5.0 hours, about 30 minutes to about 4.5 hours, about 30 minutes to about 4.0 hours, about 30 minutes to about 3.5 hours, about 30 minutes to about 3.0 hours, about 30 minutes to about 2.5 hours, about 30 minutes to about 2.0 hours, about 30 minutes to about 1.5 hours, about 30 minutes to about 1.0 hour, about 30 minutes to about 50 minutes, about 30 minutes to about 40 minutes, about 1.0 hour to about 5.0 hours, about 1.0 hour to about 4.5 hours, about 1.0 hour to about 4.0 hours, about 1.0 hour to about 3.5 hours, about 1.0 hour to about 3.0 hours, about 1.0 hour to about 2.5 hours, about 1.0 hour to about 2.0 hours, about 1.0 hour to about 1.5 hours, about 2.0 hours to about 5.0 hours, about 2.0 hours to about 4.5 hours, about 2.0 hours to about 4.0 hours, about 2.0 hours to about 3.5 hours, about 2.0 hours to about 3.0 hours, about 2.0 hours to about 2.5 hours, about 3.0 hours to about 5.0 hours, about 3.0 hours to about 4.5 hours, about 3.0 hours to about 4.0 hours, about 3.0 hours to about 3.5 hours, about 4.0 hours to about 5.0 hours, about 4.0 hours to about 4.5 hours, or about 4.5 hours to about 5.0 hours).

After permeabilization of the biological sample, the target nucleic acid in the permeabilized biological sample is contacted and hybridized with a reverse transcription primer (e.g., any of the reverse transcription primers described herein) to generate a cDNA molecule comprising a sequence that is substantially complementary to the target nucleic acid.

In some embodiments, after the generation of the cDNA molecule, a second adaptor sequence is ligated to the 3′ end of the cDNA molecule. Any suitable adaptor sequence described herein can be ligated to the 3′ end of the cDNA molecule. In some embodiments, the second adaptor molecule is a template switching oligonucleotide (TSO).

In some embodiments, a 3′ end of the cDNA molecule is extended to include a second adaptor sequence. Any adaptor sequence described herein can be included to the 3′ end of the cDNA molecule. In some embodiments, the second adaptor molecule is a template switching oligonucleotide (TSO), or a complement thereof.

The generation of the cDNA molecule and the addition (e.g., via extension) or ligation of the second adaptor sequence can be performed over about 1 minute to about 2 hours (e.g., about 1 minute to about 1.5 hours, about 1 minute to about 1.0 hour, about 1 minute to about 40 minutes, about 1 minute to about 20 minutes, about 1 minute to about 10 minutes, about 10 minutes to about 2.0 hours, about 10 minutes to about 1.5 hours, about 10 minutes to about 1.0 hour, about 10 minutes to about 40 minutes, about 10 minutes to about 20 minutes, about 20 minutes to about 2.0 hours, about 20 minutes to about 1.5 hours, about 20 minutes to about 1.0 hour, about 20 minutes to about 40 minutes, about 40 minutes to about 2.0 hours, about 40 minutes to about 1.5 hours, about 40 minutes to about 1.0 hour, about 1.0 hour to about 2.0 hours, about 1 hour to about 1.5 hours, or about 1.5 hours to about 2.0 hours).

In some embodiments, the reverse transcription occurs within the permeabilized biological sample, e.g., a permeabilized tissue sample. In some embodiments, the permeabilization and the generation of the cDNA molecule occurs while the biological sample is disposed on the array.

After the generation of the cDNA molecule and the addition (e.g., via extension) or ligation of the second adaptor sequence, the cDNA molecule can be denatured/released from the target nucleic acid and the cDNA molecule is contacted with an array comprising a capture probe. The capture probe can include in a 5′ to a 3′ direction: (i) a spatial barcode and (ii) a capture domain that binds specifically to the second adaptor sequence ligated to the cDNA. When contacting with the array with the cDNA molecule, the attached capture probe captures the cDNA molecule using the capture domain, and the 3′ end of the capture probe is extended to add a sequence that is substantially complementary to the cDNA molecule sequence. In some embodiments, the methods can further include generating a single-stranded nucleic acid that includes a sequence that is substantially complementary to the extended capture probe. The optional steps of extending a 3′ end of the capture probe (using the specifically bound cDNA as a template) and generating a single-stranded nucleic acid including a sequence complementary to the extended capture probe can be performed for about 5 minutes to about 2 hours (or any of the subranges within this range described herein).

In some embodiments, a single-stranded nucleic acid including a sequence complementary to the extended capture probe can be denatured from the extended capture probe, and optionally, transferred to a different tube or container for performance of additional steps.

The single-stranded nucleic acid including a sequence complementary to the extended capture probe can be quantitated and/or sequenced or at least partially sequenced using any of the methods described herein, or described in, e.g., Sections (II)(a) or (II)(g) (e.g., (II)(g)(iv)) of WO 2020/176788 and/or U.S. Patent Application Publication No. 2020/0277663 or known in the art. In some embodiments, the quantitation and/or sequencing of the single-stranded nucleic acid including a sequence complementary to the extended capture probe can be quantitated and/or sequenced or at least partially sequenced for about 10 minutes to about 2 hours (or any of the subranges of this range described herein).

Following the denaturing of the single-stranded nucleic acid including a sequence complementary to the extended capture probe, the single-stranded nucleic acid including the sequence complementary to the extended capture probe can be subjected to amplification, fragmentation, end-repairing, A-tailing, adaptor ligation, sample index PCR, and the construction and quality control of a gene expression library, using any of the exemplary methods described herein, or described in, e.g., Sections (II)(a) or (II)(g) (e.g., (II)(g)(iv)) of WO 2020/176788 and/or U.S. Patent Application Publication No. 2020/0277663.

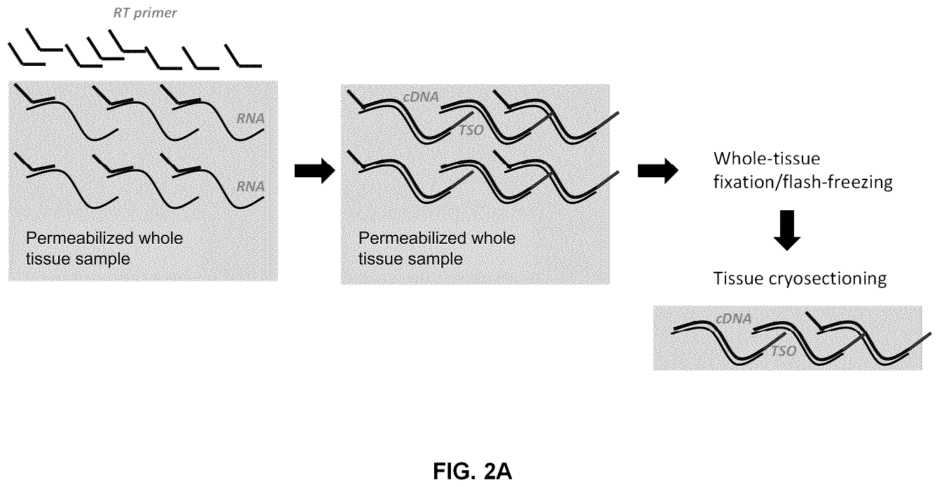

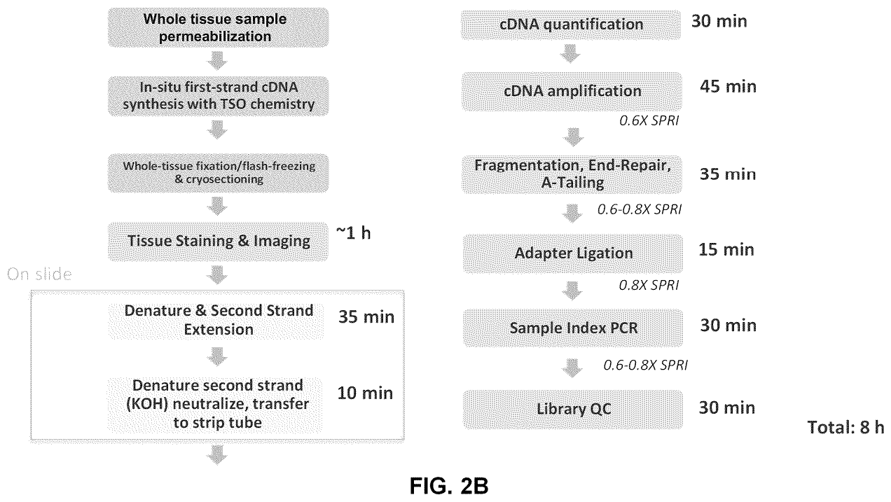

FIG. 2A is an exemplary diagram showing, from left to right, the hybridization of a reverse transcription primer to a target nucleic acid, e.g., an mRNA, within a permeabilized biological sample, e.g., a whole tissue sample that has not been sectioned, cut or further fragmented; the generation of a cDNA molecule comprising a sequence that is substantially complementary to the target nucleic acid, and the additon (e.g., via extension) or ligation of a second adaptor sequence (e.g., a template switching oligonucleotide, or a complement thereof) to the 3′ end of the cDNA molecule within the permeabilized biological sample, e.g., a whole tissue sample that has not been sectioned, cut or further fragmented; the fixation and/or flash-freezing of the biological sample, and the cryosectioning of the whole tissue sample for use in additional steps. When using a whole tissue sample that has not been sectioned, cut or further fragmented, the steps described in FIG. 2A are performed when the biological sample is not disposed on an array.

FIG. 2B is an exemplary workflow showing the generation of a gene expression library and the identification of the location of a target nucleic acid in a biological sample, for example following the target analyte capture workflow of FIG. 2A. Specifically, a biological sample, e.g., a whole tissue sample, is permeabilized. Any suitable permeabilization method described herein, or in, e.g., Section (I)(d)(ii)(13) of WO 2020/176788 and/or U.S. Patent Application Publication No. 2020/0277663, or known in the art can be used to permeabilize the whole tissue sample. In some embodiments, the biological sample is permeabilized for about 5 minutes to about 5 hours (or any of the subranges of this range described herein).

After permeabilization of the biological sample, the target nucleic acid in the permeabilized biological sample is contacted and annealed with a reverse transcription primer to generate a cDNA molecule comprising a sequence that is substantially complementary to the target nucleic acid. After synthesis of the cDNA molecule, a second adaptor sequence is added (e.g., via extension) or ligated to the 3′ end of the cDNA molecule. Any suitable adaptor sequence described in the current application can be used to ligate to the 3′ cDNA molecule. In some embodiments, the second adaptor molecule is a template switching oligonucleotide (TSO), or a complement thereof. The generation of the cDNA molecule and the addition (e.g., via extension) or ligation of the second adaptor sequence can be performed for about 10 minutes to about 2 hours (or any of the subranges of this range described herein). In some embodiments, the synthesis of the cDNA molecule occurs within a permeabilized whole tissue sample.

Following the generation of the cDNA molecule, the biological sample, e.g., the whole tissue sample, can be fixed and/or flash-frozen. Any suitable methods described herein, or in, e.g., Section (I)(d)(1)-(I)(d)(4) of WO 2020/176788 and/or U.S. Patent Application Publication No. 2020/0277663, or known in the art can be used to fix and flash-freeze the tissue sample. In some embodiments, the biological sample, e.g., the whole tissue sample is formalin-fixed and paraffin-embedded (FFPE). In some embodiments, the biological sample, e.g., whole tissue sample, is flash-frozen using liquid nitrogen. The flash-frozen tissue sample is then sectioned for future steps. In some embodiments, the sectioning is performed using cryosectioning. In some embodiments, the methods further comprise a thawing step, after the cryosectioning.

After sectioning, the biological sample, e.g., tissue sample, can be stained, and imaged. Any of the methods described herein, or in, e.g., Section (I)(d)(6) or (II)(a)(i) of WO 2020/176788 and/or U.S. Patent Application Publication No. 2020/0277663, or known in the art can be used to stain and/or image the biological sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

In some embodiments, the generation of the cDNA occurs within the permeabilized biological sample, e.g., a permeabilized tissue sample. In some embodiments, the permeabilization and the generation of the cDNA occurs while the biological sample is not disposed on the array.

After the generation of the cDNA molecule, the addition (e.g., via extension) or ligation of the second adaptor sequence to a 3′ end of the cDNA, and the tissue fixation, freezing, sectioning, staining, and imaging, the cDNA is released/denatured from the target nucleic acid and the cDNA is contacted with an array comprising a capture probe. The capture probe can include in a 5′ to a 3′ direction: (i) a spatial barcode and (ii) a capture domain that binds specifically to the second adaptor sequence ligated to the cDNA. When the array is contacted with the cDNA, the capture probe binds specifically to the cDNA via the capture domain, and the 3′ end of the capture probe is extended (using the specifically bound cDNA as a template) to add a sequence that is substantially complementary to the cDNA. The method can further include generating a single-stranded nucleic acid that is complementary to the extended capture probe. The denaturing of the cDNA from the target nucleic acid, the extension of the capture probe (using the specifically bound cDNA as a template), and optional generation of a single-stranded nucleic acid including a sequence that is complementary to the extended capture probe can be performed over 10 minutes to about 5 hours (or any of the subranges of this range described herein).

The single-stranded nucleic acid including a sequence that is complementary to the extended capture probe can be separated/denatured from the extended capture probe and optionally, transferred to a container (e.g., a strip tube) for the performance of additional steps.

The extended capture probe and/or a denatured/separated single-stranded nucleic acid including a sequence that is complementary to the extended capture probe can be quantitated and/or sequenced or at least partially sequenced using any of the methods described herein, or described in, e.g., Sections (II)(a) or (II)(g) (e.g., (II)(g)(iv)) of WO 2020/176788 and/or U.S. Patent Application Publication No. 2020/0277663 or known in the art. In some embodiments, the quantitation and/or sequencing of the extended capture probe and/or a denatured/separated single-stranded nucleic acid including a sequence that is complementary to the extended capture probe can be performed for about 10 minutes to about 5 hours (or any of the subranges of this range described herein).

Following the denaturing of the single-stranded nucleic acid including a sequence complementary to the extended capture probe, the single-stranded nucleic acid including the sequence complementary to the extended capture probe can be subjected to amplification, fragmentation, end-repairing, A-tailing, adaptor ligation, sample index PCR, and the construction and quality control of a gene expression library, using any of the exemplary methods described herein, or described in, e.g., Sections (II)(a) or (II)(g) (e.g., (II)(g)(iv)) of WO 2020/176788 and/or U.S. Patent Application Publication No. 2020/0277663.

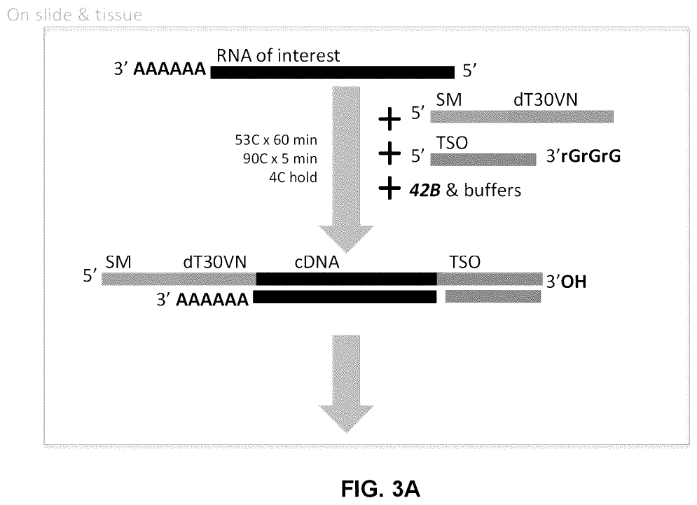

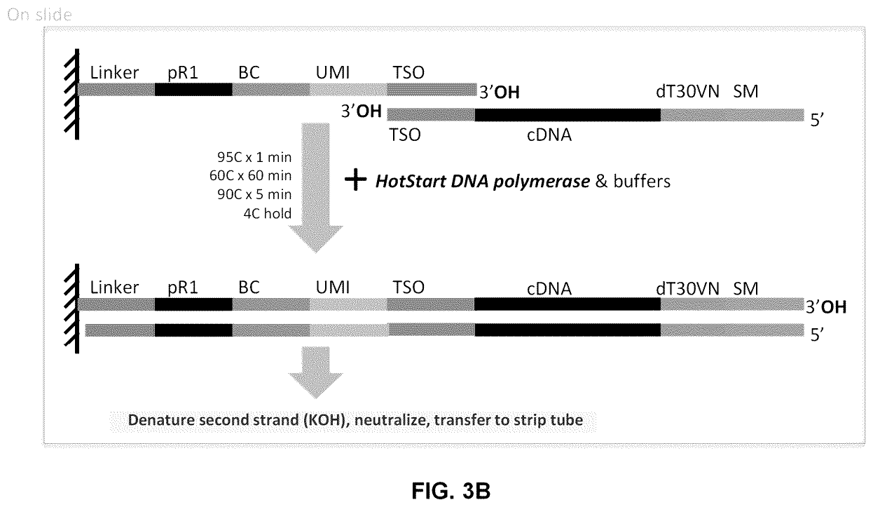

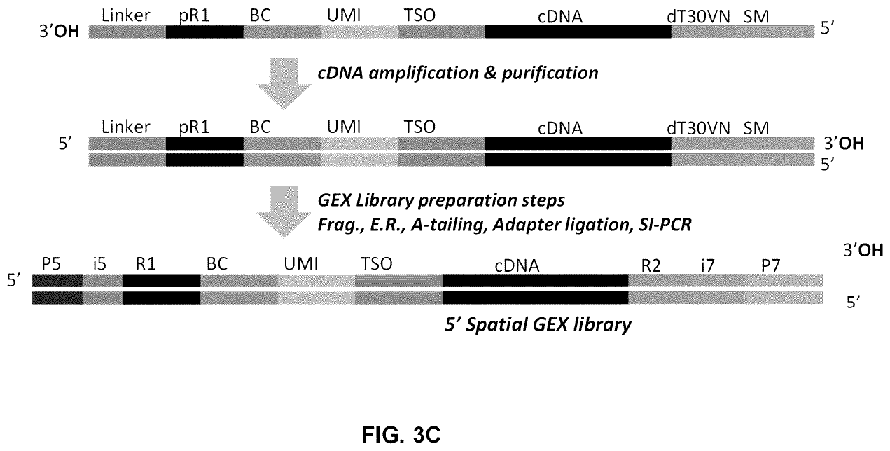

FIG. 3A is a diagram showing an exemplary reaction mix that can be used to generate a cDNA and that can be used to add (e.g., via extension) or ligate a second adaptor sequence to a 3′ end of the cDNA. For example, a target nucleic acid in the biological sample can be an mRNA molecule having a poly(A) tail at the 3′ end of the sequence. For the generation of a cDNA, a reverse transcription primer is added to the biological sample. The reverse transcription primer includes, from the 5′ end to the 3′ end, a first adaptor sequence (SM), and a sequence that is substantially complementary to a portion of the mRNA. In some embodiments, the sequence that is substantially complementary to a portion of the mRNA includes a poly(T) sequence comprising a sequence of Tₙ, wherein n can be 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, the sequence substantially complementary to a portion of the mRNA is a dT30VN sequence, where the sequence comprises Tₙ, wherein n is 30, wherein V is A, G, or C, and where N is A, G, C, or T. In some embodiments, the sequence substantially complementary to a portion of the mRNA can includes a random sequence.

In some embodiments, the complement of the second adaptor sequence can be added to a reverse transcription mix. In some embodiments, the complement of the second adaptor sequence can be a template switching oligonucleotide (TSO) having a rGrGrG sequence at the 3′ end of the second adaptor sequence.

The reverse transcription step can be performed using a method that includes a pre-equilibration thermocycling protocol (e.g., lid temperature and pre-equilibration at about 53° C., reverse transcription at about 53° C. for about 60 minutes, about 90° C. for about 5 min, and then held at about 4° C.). Any suitable reverse transcriptase and buffers can be used to perform reverse transcription, such as any of those described herein or known in the art. In some embodiments, the reverse transcription mix can further include other components that assist or increase the rate of a reverse transcription reaction. For example, the reverse transcription mix can further include dNTPs. The thermocycling protocol for the reverse transcription reaction and the ligation of the second adaptor sequence to the 3′ end of the cDNA can be further optimized according to different target nucleic acids in different types of biological samples.

The reaction described in FIG. 3A generates a cDNA comprising, for the 5′ end to the 3′ end, a first adaptor sequence (e.g., SM sequence), a sequence substantially complementary to the target nucleic acid (e.g., a dT30VN sequence), a sequence complementary to the target nucleic acid sequence, and a complement of a second adaptor sequence (e.g., a TSO sequence). In some embodiments, the cDNA is hybridized to the target nucleic acid molecule is subsequently denatured/separated from the nucleic acid analyte.

FIG. 3B is a diagram showing an exemplary array comprising a second exemplary capture probe. The capture probe comprises, from the 5′ end to the 3′ end, a linker sequence, a partial R1 primer sequence, a spatial barcode, a unique molecular identifier (UMI), a capture domain, e.g., a sequence substantially complementary to the second adaptor sequence. In some embodiments the sequence substantially complementary to the second adaptor sequence is substantially complementary to a template switching oligonucleotide (TSO) ligated to the cDNA molecule. In some embodiments the sequence substantially complementary to the second adaptor sequence is comprises a template switching oligonucleotide (TSO) used as a template for extension of a 3′ end of the cDNA molecule. In some embodiments, the 5′ end of the capture probe is attached to the array. After the capture domain on the capture probe specifically binds to the second adaptor sequence on the cDNA molecule, a 3′ end of the capture probe is extended (using the specifically bound cDNA as a template) to add a sequence that is substantially complementary to the sequence of the cDNA and a sequence complementary to the first adaptor sequence). In addition, a single-stranded nucleic acid that includes a sequence that is complementary to the extended capture probe can be generated (bottom strand shown in bottom half of figure).

The generation of the extended capture probe and the generation of the single-stranded nucleic acid that includes a sequence complementary to the extended capture probe in FIG. 3B can be performed using a thermocycling protocol (e.g., lid temperature and pre-equilibrate at about 95° C., denaturing at about 95° C. for about 1 min, reannealing at about 60° C. for about 60 min, extension at about 90° C. for about 5 minutes, and then held at about 4° C.). The reaction mixture further includes all necessary polymerase and buffers. In some embodiments, the polymerase can be a DNA polymerase. In some embodiments, the DNA polymerase can be HotStart Taq DNA polymerase.

After the generation of the single-stranded nucleic acid including a sequence that is complementary to the extended capture probe, KOH can be added to denature the single-stranded nucleic acid including a sequence complementary to the extended capture probe from the extended capture probe, and transferring the single-stranded nucleic acid including a sequence that is complementary to the extended capture probe to a different tube (e.g., one or more tubes, for example a strip tube that might be used in a thermocyling instrument) for the performance of additional steps.

FIG. 3C is a diagram showing exemplary steps of amplification, quantitation, and/or sequencing of a single-stranded nucleic acid that includes a sequence complementary to the extended capture probe. In some embodiments, the methods can include the performance of qPCR. Exemplary methods for performing qPCR are described herein and are known in the art.

In some embodiments, the method can result in the generation of a single-stranded nucleic acid that includes in a 5′ to a 3′ direction, a linker, a partial R1 primer sequence, a spatial barcode, a UMI, a sequence complementary to the second adaptor sequence, a sequence present in the target nucleic acid, and a sequence complementary to the first adaptor sequence.

In some embodiments, the method can result in the generation of a single-stranded nucleic acid that includes in a 5′ to a 3′ direction, a P5 sequencing handle, a i5 sequencing handle, a linker, a partial R1 or R1 primer sequence, a spatial barcode, a UMI, a sequence complementary to the second adaptor sequence, a sequence present in the target nucleic acid, a R2 adaptor sequence, an i7 sequencing handle, and a P7 sequencing handle.

In some embodiments, the method can result in the generation of a single-stranded nucleic acid that includes in a 3′ to a 5′ direction, a sequence complementary to a linker, a sequence complementary to a partial R1 primer sequence, a sequence complementary to a spatial barcode, a sequence complementary to a UMI, the second adaptor sequence, a sequence complementary to a sequence present in the target nucleic acid, and the first adaptor sequence.

In some embodiments, the method can result in the generation of a single-stranded nucleic acid that includes in a 3′ to a 5′ direction, a sequence complementary to a P5sequencing handle, a sequence complementary to an i5 sequencing handle, a sequence complementary to a linker, a sequence complementary to a partial R1 or R1 primer sequence, a sequence complementary to a spatial barcode, a sequence complementary to a UMI, the second adaptor sequence, a sequence complementary to a sequence present in the target nucleic acid, a sequence complementary to an R2 adaptor sequence, a sequence complementary to an i7 sequencing handle, and a sequence complementary to a P7 sequencing handle.

In some embodiments of any of the methods described herein, step (c) includes sequencing all or a part of the sequence of the spatial barcode, or a complement thereof, and sequencing all of a part of the sequence of the target nucleic acid, or a complement thereof. The sequencing can be performed using any of the methods described herein. In some embodiments, step (c) includes sequencing the full-length sequence of the spatial barcode, or a complement thereof. In some embodiments, step (c) includes sequencing a part of the sequence of the spatial barcode, or a complement thereof. In some embodiments, step (e) includes sequencing the full-length sequence of the target nucleic acid, or a complement thereof. In some embodiments, step (c) includes sequencing a part of the target nucleic acid, or a complement thereof. In some embodiments, the sequencing is performed using high throughput sequencing. In some embodiments, the target nucleic acid is sequenced from the 5′ end of the target nucleic acid. In some embodiments, the target nucleic acid is sequenced from the 3′ end of the target nucleic acid. In some embodiments, the target nucleic acid is sequenced from both the 3′ end and the 5′ end of the target nucleic acid. The library can be sequenced using available sequencing platforms, including, any of MiSeq, NextSeq 500/550, HiSeq 2500, HiSeq 3000/4000, NovaSeq, or iSeq.

### Kits

Also provided herein are kits for performing any of the methods described herein. For example, provided herein is a kit comprising: a reverse transcription primer comprising (i) a sequence that is substantially complementary to a portion of the target nucleic acid and (ii) a first adaptor sequence; a reverse transcriptase; and an oligonucleotide comprising a second adaptor sequence or a complement thereof. In some embodiments, the reverse transcriptase is a reverse transcriptase with terminal transferase activity. In some embodiments, the second adaptor sequence or complement thereof is a TSO or complement thereof. The kits can include any other buffers, enzymes, cofactors, or other components useful in the method. For example, when the method includes ligating the second adaptor sequence to the generated cDNA molecule, the kit can further include a ligase. In some embodiments, the kits can also include an array, wherein the array comprises an attached capture probe comprising in a 5′ to a 3′ direction: (i) a spatial barcode and (ii) a capture domain that binds specifically to the second adaptor sequence. In some examples, the kit can further include a permeabilizing agent. In some embodiments, the kit can further include a lipase, a protease, and/or an RNAse.

### Exemplary Embodiments

Embodiment 1 is a method of identifying a location of a target nucleic acid in a permeabilized biological sample, the method comprising:

(a) generating a cDNA molecule comprising a sequence that is substantially complementary to the target nucleic acid using a reverse transcription primer comprising (i) a sequence that is substantially complementary to a portion of the target nucleic acid and (ii) a first adaptor sequence, wherein the step of generating the cDNA molecule occurs within the permeabilized biological sample;

(b) ligating a second adaptor sequence to a 3′ end of the cDNA molecule, wherein the step of ligating is performed within the biological sample;

(c) releasing the cDNA molecule from the target nucleic acid, such that the cDNA contacts an array, wherein the array comprises an attached capture probe comprising in a 5′ to a 3′ direction: (i) a spatial barcode and (ii) a capture domain that binds specifically to the second adaptor sequence ligated to the cDNA;

(d) extending a 3′ end of the capture probe using the cDNA as a template; and

(e) determining (i) all or a part of the sequence of the target nucleic acid, or a complement thereof, and (ii) all or a part of the sequence of the spatial barcode, or a complement thereof, and using the determined sequences of (i) and (ii) to identify the location of the target nucleic acid in the permeabilized biological sample.

Embodiment 2 is a method of identifying a location of a target nucleic acid in a permeabilized biological sample, the method comprising:

(a) generating a cDNA molecule comprising a sequence that is substantially complementary to the target nucleic acid using a reverse transcription primer comprising (i) a sequence that is substantially complementary to a portion of the target nucleic acid and (ii) a first adaptor sequence, wherein the step of generating the cDNA molecule occurs within the permeabilized biological sample;

(b) extending a 3′ end of the cDNA molecule to include a second adaptor sequence, wherein the step of extending is performed within the biological sample;

(c) releasing the cDNA molecule from the target nucleic acid, such that the cDNA contacts an array, wherein the array comprises an attached capture probe comprising in a 5′ to a 3′ direction: (i) a spatial barcode and (ii) a capture domain that binds specifically to the second adaptor sequence;

(d) extending a 3′ end of the capture probe using the cDNA as a template; and

(e) determining (i) all or a part of the sequence of the target nucleic acid, or a complement thereof, and (ii) the sequence of the spatial barcode, or a complement thereof, and using the determined sequences of (i) and (ii) to identify the location of the target nucleic acid in the permeabilized biological sample.

Embodiment 3 is the method of Embodiment 2, wherein step (b) occurs simultaneously with step (a).

Embodiment 4 is the method of any one of Embodiments 1-3, wherein steps (a) through (c) are performed when the biological sample is disposed on the array.

Embodiment 5 is the method of any one of Embodiments 1-3, wherein step (a) is performed when the biological sample is not disposed on the array and step (b) is performed when the biological sample is disposed on the array, and wherein the method further comprises between steps (a) and (b), a step of disposing the biological sample on the array.

Embodiment 6 is the method of any one of Embodiments 1-3, wherein steps (a) and (b) are performed when the biological sample is not disposed on the array, and wherein the method further comprises between steps (b) and (c), a step of disposing the biological sample on the array.

Embodiment 7 is the method of any one of Embodiments 1-6, wherein the sequence that is substantially complementary to a portion of the target nucleic acid present in the reverse transcription primer comprises a poly(T) sequence.

Embodiment 8 is the method of any one of Embodiments 1-6, wherein the sequence that is substantially complementary to a portion of the target nucleic acid present in the reverse transcription primer comprises a random sequence.

Embodiment 9 is the method of any one of Embodiments 1-8, wherein the second adaptor sequence is a template switching oligonucleotide (TSO), or a complement thereof.

Embodiment 10 is the method of any one of Embodiments 1-9, wherein the array comprises a slide.

Embodiment 11 is the method of Embodiment 10, wherein a 5′ end of the capture probe is attached to the slide.

Embodiment 12 is the method of any one of Embodiments 1-9, wherein the array is a bead array.

Embodiment 13 is the method of Embodiment 12, wherein a 5′ end of the capture probe is attached to a bead of the bead array.

Embodiment 14 is the method of any one of Embodiments 1-13, wherein the capture probe further comprises a unique molecular identifier (UMI).

Embodiment 15 is the method of Embodiment 14, wherein the UMI is positioned 5′ relative to the capture domain in the capture probe.

Embodiment 16 is the method of any one of Embodiments 1-15, wherein the determining in step (e) comprises sequencing (i) all or a part of the sequence of the target nucleic acid, or a complement thereof, and (ii) the sequence of the spatial barcode, or a complement thereof.

Embodiment 17 is the method of Embodiment 16, wherein the sequencing is high throughput sequencing.

Embodiment 18 is the method of Embodiment 16, wherein the sequencing is sequencing by hybridization.

Embodiment 19 is the method of any one of Embodiments 1-18, wherein the target nucleic acid is RNA.

Embodiment 20 is the method of Embodiment 19, wherein the RNA is an mRNA. Embodiment 21 is the method of any one of Embodiments 1-20, wherein the permeabilized biological sample is a permeabilized tissue section.

Embodiment 22 is the method of Embodiment 21, wherein the permeabilized tissue section is a permeabilized formalin-fixed and paraffin-embedded (FFPE) tissue section.

Embodiment 23 is the method of any one of Embodiments 1-22, wherein the method further comprises a step of imaging the biological sample.

Embodiment 24 is the method of Embodiment 23, wherein the step of imaging is performed prior to step (a).

Embodiment 25 is the method of Embodiment 24, wherein the step of imaging is performed between steps (b) and (c).

Embodiment 26 is the method of any one of Embodiments 1-3 and 6-25, wherein the method further comprises, between steps (b) and (c), a step of freezing and thawing the permeabilized biological sample.

Embodiment 27 is the method of Embodiment 26, wherein the method further comprises, between steps (b) and (c), a step of sectioning the permeabilized biological sample.

Embodiment 28 is the method of Embodiment 27, wherein the step of sectioning the permeabilized biological sample is performed using cryosectioning.

Embodiment 29 is the method of any one of Embodiments 1-28, wherein the method further comprises, prior to step (a), a step of permeabilizing the biological sample.

Embodiment 30 is the method of any one of Embodiments 1-29, wherein the performance of step (a) comprises introducing a reverse transcriptase, dNTPs, and the reverse transcription primer into the permeabilized biological sample.

Embodiment 31 is a kit comprising: a reverse transcription primer comprising (i) a sequence that is substantially complementary to a portion of the target nucleic acid and (ii) a first adaptor sequence; a reverse transcriptase; and an oligonucleotide comprising a second adaptor sequence or a complement thereof.

Embodiment 32 is the kit of Embodiment 31, wherein the kit further comprises a ligase.

Embodiment 33 is the kit of Embodiment 30 or 31, wherein the reverse transcriptase is a reverse transcriptase with terminal transferase activity.

Embodiment 34 is the kit of any one of Embodiments 31-33, wherein the second adaptor sequence or the complement thereof is a TSO or a complement thereof.

Embodiment 35 is the kit of any one of Embodiments 31-34, wherein the kit further comprises an array, wherein the array comprises an attached capture probe comprising in a 5′ to a 3′ direction: (i) a spatial barcode and (ii) a capture domain that binds specifically to the second adaptor sequence.

Embodiment 36 is a nucleic acid comprising, in the 5′ to 3′ direction: a spatial barcode; a sequence complementary to a second adaptor sequence; a sequence present in a target nucleic acid; and a sequence complementary to a first adaptor sequence.

Embodiment 37 is a nucleic acid comprising, in the 3′ to 5′ direction: a complement of a spatial barcode; a second adaptor sequence; a sequence complementary to a sequence present in a target nucleic acid; and a first adaptor sequence.

Embodiment 38 is the nucleic acid of Embodiment 36 or 37, wherein the second adaptor sequence comprises a TSO.

Embodiment 39 is the nucleic acid of Embodiment 36 or 37, wherein the second adaptor sequence comprises a complement of a TSO.

Embodiment 40 is the nucleic acid of any one of Embodiments 36-39, wherein the first adaptor sequence is a reverse transcriptase primer.

What is claimed is:

1. A method of identifying a location of a target nucleic acid in a permeabilized tissue section, the method comprising:
(a) generating a cDNA molecule comprising a sequence that is substantially complementary to the target nucleic acid using a reverse transcription primer comprising (i) a sequence that is substantially complementary to a portion of the target nucleic acid and (ii) a first adaptor sequence, wherein the step of generating the cDNA molecule occurs within the permeabilized tissue section;
(b) ligating a second adaptor sequence to a 3′ end of the cDNA molecule, wherein the step of ligating is performed within the tissue section;
(c) releasing the cDNA molecule ligated to the second adaptor sequence from the target nucleic acid in the tissue section, such that the second adaptor sequence contacts an array, wherein the array comprises an attached capture probe comprising in a 5′ to a 3′ direction: (i) a spatial barcode and (ii) a capture domain that hybridizes to the second adaptor sequence ligated to the cDNA molecule;
(d) extending a 3′ end of the capture probe using the cDNA molecule as a template; and
(e) determining (i) all or a part of the sequence of the target nucleic acid, or a complement thereof, and (ii) the sequence of the spatial barcode, or a complement thereof, and using the determined sequences of (i) and (ii) to identify the location of the target nucleic acid in the permeabilized tissue section.
